(19) United States
(12) Patent Application Publication
(10) Pub. No.: US 2022/0168253 A1
(43) Pub. Date: Jun. 2, 2022

(54) PRODUCTION PROMOTER OF TYPE I COLLAGEN OR ELASTIN

(71) Applicant: DR'S CHOICE CO., LTD.

(72) Inventors: Masahiko NAKANO, Chiyoda-ku (JP); Akio YAMAMOTO, Chiyoda-ku (JP)

(73) Assignee: DR'S CHOICE CO., LTD., Chiyoda-ku (JP)

(21) Appl. No.: 17/432,327

(22) PCT Filed: Feb. 21, 2019
(86) PCT No.: PCT/JP2019/006547

§ 371 Date: Aug. 19, 2021

(51) Int. Cl. A61K 31/198; A61K 36/02; A23L 33/175

(52) U.S. Cl. CPC A61K 31/198; A23L 33/175; A61K 36/02

(57) ABSTRACT

A type I collagen or elastin production promoter containing a mycosporine-like amino acid or a salt thereof as an active ingredient; and a method for promoting type I collagen or elastin production, the method including administering an effective amount of a mycosporine-like amino acid or a salt thereof.

### BACKGROUND OF THE INVENTION

Type I collagen, a fibrillar collagen, is abundant in bones and skins and responsible for bone and skin elasticity. Elastin is a fiber that has a role of supporting collagen fibers. The elastin content of the human skin dermis is said to be about 2% to 5%. The elastin content decreases with age to cause winkles. To prevent or reduce winkles or other signs, type I collagen or elastin is applied or orally administered.

However, it is unclear whether type I collagen or elastin in foods or cosmetics is efficiently absorbed and reaches the site of action.

Mycosporine-like amino acids, a family of amino acids found in algae, scallops, and other organisms, are known to have an effect of absorbing ultraviolet radiation (Non Patent Literature 1) and an effect of promoting fibroblast growth (Patent Literature 1). Mycosporine-like amino acids are also known to have an effect of promoting hyaluronic acid production (Patent Literature 2).

### CITATION LIST

### Patent Literature

Patent Literature 1: JP 2007-16004 A

Patent Literature 2: JP 6272814 B1

### Non Patent Literature

Non Patent Literature 1: Phycol. 34, 418-430 (1998)

### SUMMARY OF THE INVENTION

### Technical Problem

Since it is unclear whether type I collagen or elastin is absorbed and efficiently reaches the site of action as described above, there is a need of a type I collagen or elastin production promoter for producing type I collagen or elastin by using a highly absorbable natural ingredient.

An object of the present invention is to provide a novel type I collagen or hyaluronic acid production promoter.

### Solution to Problem

The inventors of the present invention studied the functions of various naturally derived ingredients in order to find a novel type I collagen or elastin production promoter and found that mycosporine-like amino acids or salts thereof have a strong effect of promoting type I collagen and elastin production and that the effect is so strong that it cannot be expected from the fibroblast growth promoting effect, thereby completing the present invention.

Specifically, the present invention relates to the following aspects (1) to (16).

(1) A type I collagen or elastin production promoter comprising a mycosporine-like amino acid or a salt thereof as an active ingredient.

(2) The type I collagen or elastin production promoter according to (1), wherein the mycosporine-like amino acid or the salt thereof is derived from algae.

(3) A type I collagen gene or elastin gene expression promoter comprising a mycosporine-like amino acid or a salt thereof as an active ingredient.

(4) The type I collagen gene or elastin gene expression promoter according to (3), wherein the mycosporine-like amino acid or the salt thereof is derived from algae.

(5) Use of a mycosporine-like amino acid or a salt thereof for manufacture of a type I collagen or elastin production promoter.

(6) The use according to (5), wherein the mycosporine-like amino acid or the salt thereof is derived from algae.

(7) Use of a mycosporine-like amino acid or a salt thereof for manufacture of a type I collagen gene or elastin gene expression promoter.

(8) The use according to (7), wherein the mycosporine-like amino acid or the salt thereof is derived from algae.

(9) A mycosporine-like amino acid or a salt thereof for promoting type I collagen or elastin production.

(10) The compound according to (9), wherein the mycosporine-like amino acid or the salt thereof is derived from algae.

(11) A mycosporine-like amino acid or a salt thereof for promoting type I collagen gene or elastin gene expression.

(12) The compound according to (11), wherein the mycosporine-like amino acid or the salt thereof is derived from algae.

(13) A method for promoting type I collagen or elastin production, the method comprising administering an effective amount of a mycosporine-like amino acid or a salt thereof.

(14) The method according to (13), wherein the mycosporine-like amino acid or the salt thereof is derived from algae.

(15) A method for promoting type I collagen gene or elastin gene expression, the method comprising administering an effective amount of a mycosporine-like amino acid or a salt thereof.

(16) The method according to (15), wherein the mycosporine-like amino acid or the salt thereof is derived from algae.

### Advantageous Effects of the Invention

A type I collagen or elastin production promoter according to the present invention, which is easily absorbed due to its low molecular weight, produces type I collagen or elastin in vivo after being administered transdermally or orally. The type I collagen or elastin production promoter thus promotes type I collagen or elastin production in the joints, skins, and other organs and is useful as, for example, pharmaceuticals, cosmetics, and functional foods in order to, for example, relieve joint pain, improve joint movement, and improve skin elasticity.

### BRIEF DESCRIPTION OF THE DRAWINGS

FIG. 1 is a graph illustrating the effect of mycosporine-like amino acids (MAAs) in promoting elastin gene expression.

FIG. 2 is a graph illustrating the effect of mycosporine-like amino acids (MAAs) in promoting type I collagen gene expression.

### DETAILED DESCRIPTION OF THE INVENTION

An active ingredient of a type I collagen or elastin production promoter or a type I collagen or elastin gene expression promoter according to the present invention is a mycosporine-like amino acid or a salt thereof.

Mycosporine-like amino acids are amino acids having a cyclohexanone structure and known to be produced by algae such as seaweed and microalgae, marine products, and fungi, and other organisms. Mycosporine-like amino acids have been found as ultraviolet absorbing substances. Although mycosporine-like amino acids are known to have an effect of absorbing ultraviolet radiation and an effect of promoting fibroblast growth as described above, they are not known to have an effect of promoting type I collagen and elastin production.

The following compounds are known as examples of mycosporine-like amino acids.

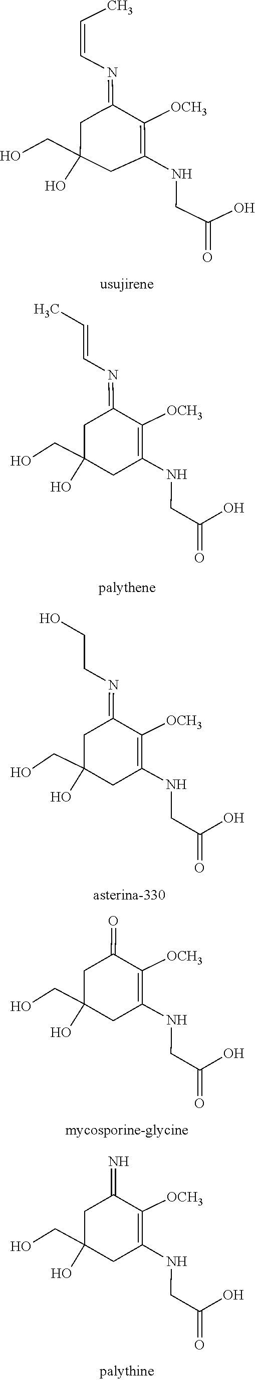 [SMILES: C/C=C\N=C1/CCC(O)(CO)CC(NCC(=O)O)=C1OC ; C/C=C/N=C1\CC(O)(CO)CC(NCC(=O)O)=C1OC ; COC1=C(NCC(=O)O)CC(O)(CO)C/C1=N\CCO ; COC1=C(NCC(=O)O)CC(O)(CO)CC1=O ; COC1=C(NCC(=O)O)CC(O)(CO)CC1=N]

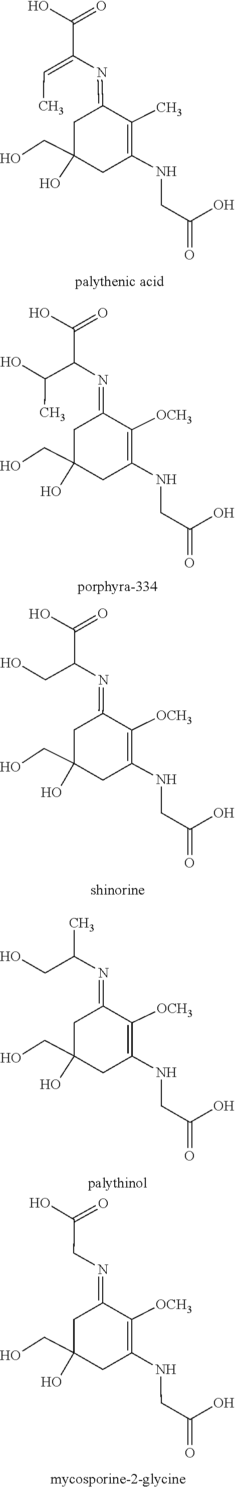 [SMILES: C/C=C(\N=C1/CC(O)(CO)CC(NCC(=O)O)=C1C)C(=O)O ; COC1=C(NCC(=O)O)CC(O)(CO)C/C1=N\C(C(=O)O)C(C)O ; COC1=C(NCC(=O)O)CC(O)(CO)C/C1=N\C(CO)C(=O)O ; COC1=C(NCC(=O)O)CC(O)(CO)C/C1=N\C(C)CO ; COC1=C(NCC(=O)O)CC(O)(CO)C/C1=N\CC(=O)O]

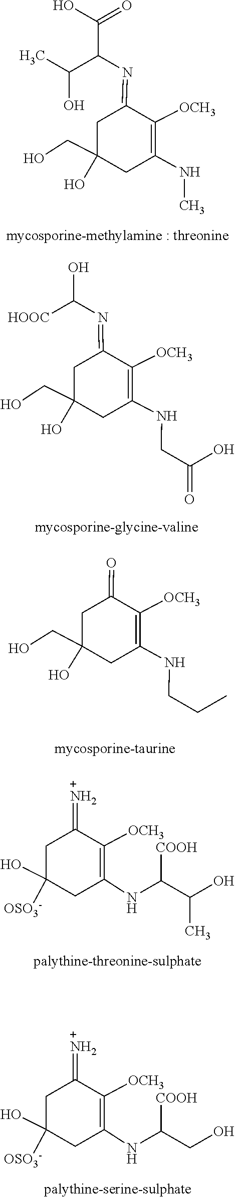

Mycosporine-like amino acids or salts thereof used in the present invention can be extracted from, for example, marine products such as shellfish, and fungi, but preferably derived from algae, more preferably derived from blue-green algae, red algae, brown algae, or green algae. Mycosporine-like amino acids derived from algae include the above compounds.

To extract mycosporine-like amino acids from algae or shellfish, a polar solvent, such as water, ethanol, acetone, methanol, isopropanol, acetonitrile, or a mixed solvent thereof, can be used to extract mycosporine-like amino acids from dried or freeze-dried algae. The extraction solvent is preferably a water-ethanol mixture and more preferably a mixture of water:ethanol=1:9 to 9:1. The obtained extract is preferably concentrated by adsorbing mycosporine-like amino acids to an activated-carbon adsorption column, a resin column, or other columns, and eluting them with a water-ethanol mixture. The mycosporine-like amino acids can be purified by high performance liquid chromatography.

An algal extract containing mycosporine-like amino acids is already commercially available, and such a commercially available extract can also be used.

Examples of salts of mycosporine-like amino acids include acid addition salts, such as hydrochlorides, and alkali metal salts, such as sodium salts and potassium salts.

Mycosporine-like amino acids or salts thereof have a strong effect of promoting type I collagen and elastin production as described below in Examples. Such a preferable effect of promoting type I collagen and elastin production is so strong that it cannot be expected from the fibroblast growth promoting effect of mycosporine-like amino acids.

Mycosporine-like amino acids or salts thereof have an effect of strongly promoting type I collagen gene or elastin gene expression. The effect of mycosporine-like amino acids in promoting type I collagen or elastin production is thus considered to be based on the effect of promoting the type I collagen gene or elastin gene expression.

The type I collagen or elastin production promoter according to the present invention has an effect of promoting type I collagen or elastin production in various sites in vivo. The type I collagen or elastin production promoter according to the present invention is thus useful as, for example, pharmaceuticals, quasi-drugs, cosmetics, foods for specified health uses, foods with functional claims, functional foods, beauty foods, and foods for sick people in order to provide, for example, drugs for relieving joint pain or improving joint movement and drugs for improving skin elasticity.

Examples of the dosage form of the type I collagen or elastin production promoter according to the present invention include oral dosage forms, injection dosage forms, and transdermal dosage forms. For use as, for example, pharmaceuticals, quasi-drugs, or foods with functional claims, the type I collagen or elastin production promoter according to the present invention is preferably in the form of preparations for oral administration, such as tablets, granules, fine granules, capsules, and troches; or skin external preparations, such as creams, ointments, emulsions, and lotions. For use as foods for specified health uses or foods with functional claims, the type I collagen or elastin production promoter according to the present invention may be in the same form as common foods, such as beverages and yogurt.

The amount of a mycosporine-like amino acid or a salt thereof in such a preparation is preferably, but not necessarily, from 0.001 to 50 mass %, more preferably from 0.001 to 30 mass %.

The preparations for oral administration can further contain, for example, an excipient, a binder, a disintegrant, a lubricant, a colorant, a corrigent, and a deodorant, in addition to the mycosporine-like amino acid or the salt thereof. The skin external preparations can further contain, for example, various oils, a coloring agent, a preservative, a surfactant, and a thickener, in addition to the mycosporine-like amino acid or the salt thereof. The skin external preparations can further contain a hyaluronic acid production promoter.

### EXAMPLES

Next, the present invention will be more specifically described by way of Examples.

### Text Example 1 (Epidermal Cytotoxicity Test)

### 1) Conditions for Culturing Human Normal Skin-Derived Epidermal Cells

Human normal skin-derived epidermal cells were cultured by using HuMedia-KG2 (medium for epidermal cells) in a CO₂ incubator (5% CO₂, 37° C.). The medium was exchanged every other day.

### 2) Cytotoxicity Test

Normal human epidermal cells were seeded in a 96-well plate at a concentration of 2×10⁴ cells/100 μL/well. The next day, the medium was replaced with media (100 μL/well) having different sample concentrations (10-fold dilution series with 10% being the highest concentration, 8 levels of concentration), followed by 24-hour culturing. The cell morphology was observed under a phase-contrast microscope at the end of culturing, and the medium was removed, followed by washing with PBS once. A medium (100 μL) supplemented with 10% of viable cell count reagent SF was then added, followed by incubation at 37° C. in 5% CO₂ for 90 minutes. During incubation, the absorbance of the sample was measured by using a microplate reader every 30 minutes (measurement wavelength: 450 nm, control wavelength: 595 nm). The cell viability was calculated as O.D. values after 60 minutes to 0 minutes. The cell viability was expressed as a relative value when the cell viability in the negative control test section was defined as 100%. The test was carried out at n=3 for each test concentration.

### 3) Results

No cytotoxicity was observed even after the cells were cultured in the presence of 0.00001 mg/mL to 10 mg/mL of mycosporine-like amino acids (dried product of mycosporine-like amino acids extracted and fractionated with a 70% ethanol aqueous solution and an activated carbon column from algae, as in JP 2007-16004 A) serving as samples.

### Test Example 2 (Fibroblast Growth Test)

### 1) Culture Conditions

Normal human fibroblasts were cultured by using a 10% FBS-supplemented D-MEM in a CO₂ incubator (5% CO₂, 37° C.) The 10% FBS-supplemented D-MEM was prepared by adding 50 mL of FBS and 5 mL of penicillin-streptomycin solution to 445 mL of D-MEM.

### 2) Cell Activation Test (WST-8 Assay)

Fibroblasts prepared at 5×10⁴ cells/mL in a growth medium were seeded at 100 μL per well (96-well plate) (5×10³ cells/100 μL/well). This is the number of cells to be approximately 50% confluent and in the logarithmic growth phase the next day, which is suitable for analyzing the effect of a test substance on the cell growth activation. The fibroblasts were cultured for about 24 hours after seeding, and the growth medium was replaced with 100 μL of growth media having different sample concentrations, followed by 24-hour culturing. The media were removed at the end of culturing, and 100 μL of a medium supplemented with 10% of viable cell count reagent SF was added, followed by incubation at 37° C. in 5% CO₂ (measurement wavelength: 450 nm, control wavelength: 595 nm). After addition, the absorbance was measured at 30 minutes and 90 minutes by using a microplate reader (measurement: 450 nm), and the change in absorbance per hour was calculated from the absorbances at 30 minutes and 90 minutes. The test was carried out at n=5 for each test concentration.

### 3) Results

The relative viable cell count in the group with addition of 1 mg/mL of mycosporine-like amino acids was 109±2.22%, which was about 10% higher than 100±1.43% in the negative control group and higher than that in the negative control group with a significance (p<0.05).

TABLE 1
Cell Viability(%)

| Relative | Negative | MAAs(mg/mL) | | | | | |
|---|---|---|---|---|---|---|---|
| value (%) | control | 0.0016 | 0.008 | 0.04 | 0.2 | 1 | 10% FBS |
| | 101 | 89 | 86 | 86 | 96 | 105 | 122 |
| | 102 | 86 | 91 | 90 | 97 | 104 | 108 |
| | 102 | 86 | 89 | 96 | 94 | 111 | 100 |
| | 100 | 81 | 87 | 85 | 102 | 116 | 99 |
| | 94 | 86 | 74 | 77 | 72 | 107 | 100 |
| Mean | 100 | 86 | 86 | 87 | 94 | 109 | 106 |
| Standard | 1.43 | 1.22 | 2.91 | 3.05 | 3.94 | 2.22 | 4.36 |
| deviation | | | | | | | |

### Test Example 3 (Gene Expression Test)

The medium used was D-MEM (Sigma-Aldrich)+10% FBS (Biowest), and human normal fibroblasts (HDFn, Gibco) were seeded at 9.0×10⁴/well (12-well plate). After colonization, the medium was replaced with a starvation medium (D-MEM (Sigma-Aldrich)) for 24 hours. Subsequently, the medium was replaced with D-MEM prepared so as to contain 0, 10, 25, 30, 40, and 50 μg/mL of mycosporine-like amino acids. The cells were collected after 3 hours, and the RNA was extracted by using a RNA extraction kit (ReliaPrep™ RNA Miniprep Systems, catalog no. Z6012, Promega Corporation). The cDNA was then synthesized (Toyobo Co., Ltd., product number FSQ-301S), and the type I collagen gene or elastin gene expression was analyzed by real-time RT-PCR (SYBR Fast qPCR Mix, catalog no. RR430B, Takara Bio Inc.).

The following primers were used. To normalize the gene expression, the gene expression level was corrected with the expression level of the Ribosomal Protein Lateral Stalk Subunit P0 (RPLPO) gene.

TABLE 2

| Primer | Sequence |
|---|---|
| Elastin | Forward 5′-AGGTGTATACCCAGGTGGCGTGCT-3′ (SEQ ID NO: 1) |
| | Reverse 5′-CAACCCCTGTCCCTGTTGGGTAAC-3′ (SEQ ID NO: 2) |
| Type I | Forward 5′-CTGGTCCCCAAGGCTTCCAAGGTC-3′ (SEQ ID NO: 3) |
| collagen | Reverse 5′-CCATCATTTCCACGAGCACCAGCA-3′ (SEQ ID NO: 4) |
| RPLP0 | Forward 5′-TTCGACAATGGCAGCATCTACAA-3′ (SEQ ID NO: 5) |
| | Reverse 5′-CTGCAGACAGACACTGGCAACA-3′ (SEQ ID NO: 6) |

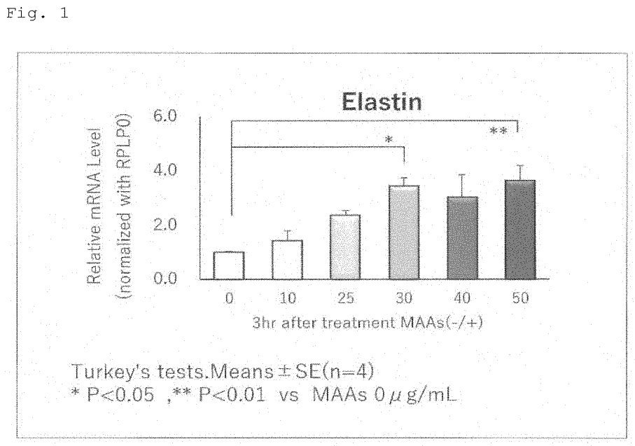

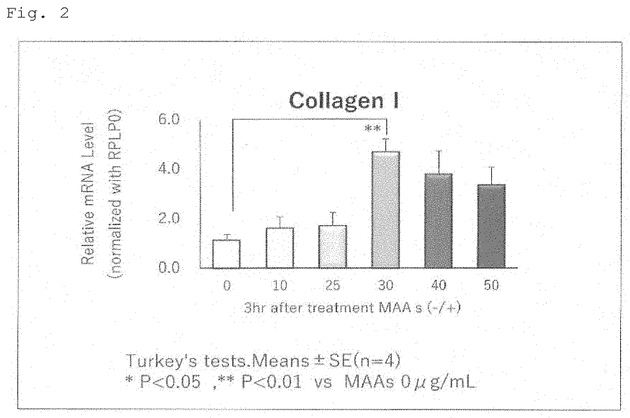

As a result, the elastin gene expression and the type I collagen gene expression were both significantly high at 30 μg/mL of mycosporine-like amino acids as shown in FIG. 1 and FIG. 2.

1-12. (canceled)

13. A method for promoting type I collagen or elastin production, the method comprising administering an effective amount of a mycosporine-like amino acid or a salt thereof.

14. The method according to claim 13, wherein the mycosporine-like amino acid or the salt thereof is derived from algae.

15. A method for promoting type I collagen gene or elastin gene expression, the method comprising administering an effective amount of a mycosporine-like amino acid or a salt thereof.

16. The method according to claim 15, wherein the mycosporine-like amino acid or the salt thereof is derived from algae.


SEQUENCE LISTING (6 sequences)

SEQ ID NO: 1 - DNA, 24 bp, Artificial Sequence
  Designed DNA bsaed on Elastin gene
aggtgtatacccaggtggcgtgct

SEQ ID NO: 2 - DNA, 24 bp, Artificial Sequence
  Designed DNA bsaed on Elastin gene
caacccctgtccctgttgggtaac

SEQ ID NO: 3 - DNA, 24 bp, Artificial Sequence
  Designed DNA bsaed on Type I Collagen gene
ctggtccccaaggcttccaaggtc

SEQ ID NO: 4 - DNA, 24 bp, Artificial Sequence
  Designed DNA bsaed on Type I Collagen gene
ccatcatttccacgagcaccagca

SEQ ID NO: 5 - DNA, 23 bp, Artificial Sequence
  Designed DNA bsaed on RPLPO gene
ttcgacaatggcagcatctacaa

SEQ ID NO: 6 - DNA, 22 bp, Artificial Sequence
  Designed DNA bsaed on RPLPO gene
ctgcagacagacactggcaaca